SEQUENCE LISTING (22 sequences)

SEQ ID NO: 1 - DNA, 20 bp, Juglans nigra
ccatgaaacttcatgcgttg

SEQ ID NO: 2 - DNA, 20 bp, Juglans nigra
tccccctgaaatcttctcct

SEQ ID NO: 3 - DNA, 20 bp, Juglans nigra
aaccctacaacgccttgatg

SEQ ID NO: 4 - DNA, 20 bp, Juglans nigra
ctcggtaagccacaccaatt

SEQ ID NO: 5 - DNA, 20 bp, Juglans nigra
aaaccacctaaaaccctgca

SEQ ID NO: 6 - DNA, 20 bp, Juglans nigra
acccatctttcacgtgtgtg

SEQ ID NO: 7 - DNA, 20 bp, Juglans nigra
cttgtaatcgccctctgctc

SEQ ID NO: 8 - DNA, 20 bp, Juglans nigra
ggagaggaaaggaatccaaa

SEQ ID NO: 9 - DNA, 20 bp, Juglans nigra
ttagttagcaaacccacccg

SEQ ID NO: 10 - DNA, 20 bp, Juglans nigra
agggcactcccttatgaggt

SEQ ID NO: 11 - DNA, 19 bp, Juglans nigra
tgccgacactcctcacttc

SEQ ID NO: 12 - DNA, 18 bp, Juglans nigra
catcccaagcgaaggttg

SEQ ID NO: 13 - DNA, 20 bp, Juglans nigra
ttctcgtggtgcttgttgag

SEQ ID NO: 14 - DNA, 18 bp, Juglans nigra
tgctcaggctccacttcc

SEQ ID NO: 15 - DNA, 20 bp, Juglans nigra
acgggcagtgtatgcatgta

SEQ ID NO: 16 - DNA, 20 bp, Juglans nigra
acccatccatgatcttccaa

SEQ ID NO: 17 - DNA, 20 bp, Juglans nigra
tgcctaattagcaatttcca

SEQ ID NO: 18 - DNA, 20 bp, Juglans nigra
tacctgcaacccgttacaca

SEQ ID NO: 19 - DNA, 20 bp, Juglans nigra
ttgaacaaaaggccgttttc

SEQ ID NO: 20 - DNA, 20 bp, Juglans nigra
agatgcacagaccaaccctc

SEQ ID NO: 21 - DNA, 20 bp, Juglans nigra
cagtctcattccctttttcc

SEQ ID NO: 22 - DNA, 18 bp, Juglans nigra
cgtgatgtacgacggctg

(19) United States
(12) Plant Patent Application Publication
(10) Pub. No.: US 2006/0010538 P1
(43) Pub. Date: Jan. 12, 2006

(54) Black walnut tree named "Beineke 13"

(50) JUGLANS NIGRA; BLACK WALNUT

(76) Inventor: Walter F. Beineke, West LaFayette, IN (US)

Correspondence Address: BARNES & THORNBURG, LLP, P.O. BOX 2786, CHICAGO, IL, 60690-2786, US

(21) Appl. No.: 10/888,759

(22) Filed: Jul. 9, 2004

(51) Int. Cl. A01H 5/00

(52) U.S. Cl. PLT/154

(57) ABSTRACT

A new and distinct cultivar of black walnut tree (Juglans nigra L.) which is distinctly characterized by extremely rapid growth rate, excellent central stem tendency, and excellent straightness thereby producing good timber qualities. The culture has good resistance to anthracnose leaf disease. This new variety of black walnut trees was discovered by the applicant near South Raub, Tippecanoe County, Ind. in a black walnut planting from previously selected trees for outstanding timber production potential. This selection has been designated as BW510, a seedling progeny of unpatented BW95 in records maintained by the applicant on the performance of this selection, and grafts made from the selection and will be known henceforth as Beineke 13.

### LATIN NAME OF THE GENUS AND SPECIES

Juglans nigra L.

### BACKGROUND OF THE INVENTION

This new variety of black walnut tree (Juglans nigra L.) was discovered by the applicant near South Raub, Tippecanoe County, Ind. in a black walnut planting of seedling progeny from previously selected trees for outstanding timber producing potential. This selection has been designated as BW510, a seedling progeny of BW95, unpatented, in records maintained by the applicant on the performance of this selection, and grafts made from the selection and will be known henceforth as ‘Beineke 13.’ The male parent is unknown, as is generally the case with black walnut trees. (Beineke, 1989)

### SUMMARY OF THE INVENTION

A new and distinct cultivar of black walnut tree (Juglans nigra L.) which is distinctly characterized by extremely rapid growth rate, excellent strong central stem tendency, and excellent straightness, thereby producing excellent timber qualities, the trait of commercial interest and good resistance to anthracnose leaf disease. Beineke 13 was 9 years old when described at a location near South Raub, Ind.

After the original clone was selected, and assigned an identity number of BW510 the aforesaid tree was reproduced by collecting scions from it and grafting these onto common black walnut rootstocks at American Forestry Technologies, Inc., West Point, Ind. These asexual reproductions ran true to the originally discovered tree and to each other in all respects.

Color values used were from the Munsell Color Chart for Plant Tissues. However, color is too dependent on weather conditions and fertilization to be consistent or distinctive. For example, leaves can be made a deeper green by applying nitrogen. Walnut tree leaves turn yellow as the season progresses, especially if there is a lack of rainfall. As black walnut meats dry, they become darker. Simply being on the ground for a week causes the outer shell to darken. Bark color involves many shades of gray through brown and black.

Beineke 13 is hardy in USDA zones 4,5,6,7, and 8.

### DESCRIPTION OF THE DRAWINGS

FIG. 1 is a photograph showing the timber form of ‘Beineke 13.’

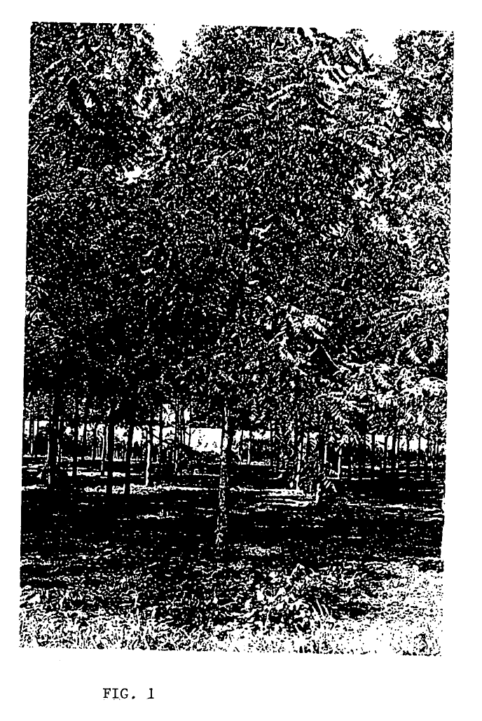

FIG. 2 is a photograph showing the leaves of ‘Beineke 13.’

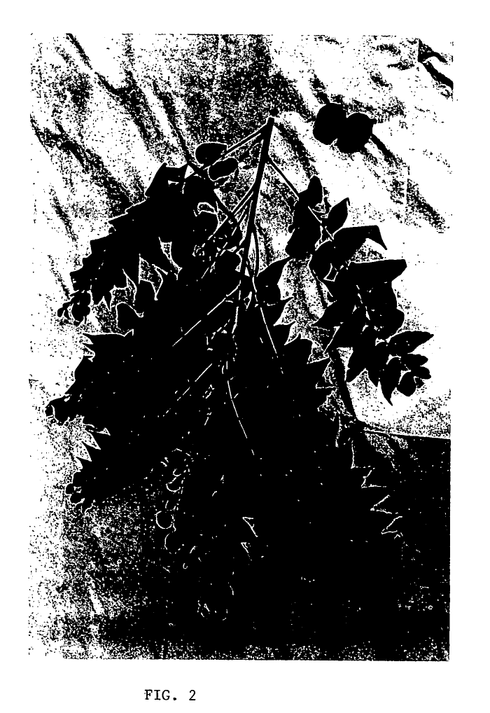

FIG. 3 is a photograph showing the nuts of ‘Beineke 13.’

### BOTANICAL DESCRIPTION OF THE PLANTS

The botanical details of this new and distinct variety of walnut tree are as follows:
Tree:
   Size.—Large, 42 ft. at 11 years; crown diameter of 20 ft.
   Vigor.—Vigorous.
   Growth rate.—Very rapid, 40.8% larger in diameter than the average of Purdue 1 (U.S. Plant Pat. No. 4,543) grafts, planted the same year on the same land. Diameter growth rate (at 4½ feet above the ground) at 11 years was 8.8 inches for an average growth rate of 0.80 inches per year.
   Form.—Excellent timber form (form rating). 59% better than the average of the entire planting. Stem form was obtained by subjectively rating the straightness of the main stem on a scale of 1 to 5 with 1 representing a perfectly straight stem; 2, slight crook or deviation of the central stem (no crooks); 3, about average straightness; 4, several severe crooks or a single fork; and 5, a very crooked, forked and/or leaning central stem. Beineke 13 averages 1 on the 1 to 5 scale.

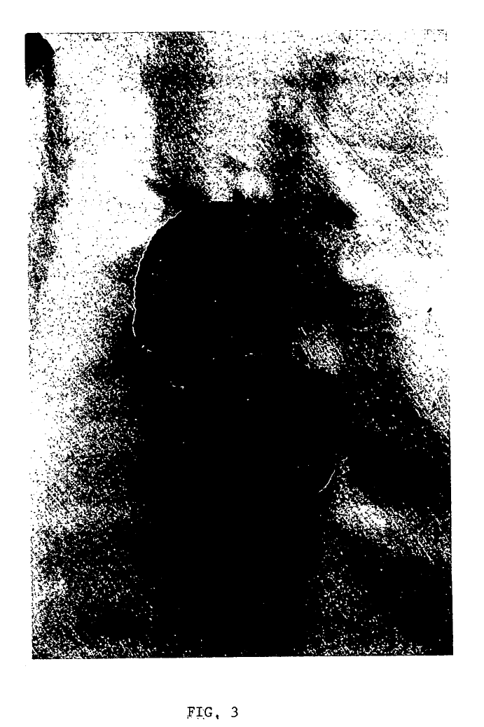

The trees of the present invention are grown in plantations, not in open fields (not natural stands). In plantations, trees are upright and have no distinctive or characteristic crown shape because all branches are seeking to grow upwards.
Branches: Diameter depends on age and size of tree, varies from ½″ to 12″, bark color varies from grays to browns.
Leaves:
   Compound leaves.—Size — Large; average length — 22.30″; width 9.93″.
   Leaflets.—Size — Large; average length — 5.08″; average width 2.20″, average number of leaflets — 21.4 — lanceolate; acutely pointed.
   Thickness.—thin; Texture — smooth; Margin — serrated; Petioles — short; Color — Topside — dark green (5GY3/4 by the Munsell Color Chart for Plant Tissues); Underside — light green (5GY5/4 on the Munsell Color Chart for Plant Tissues).
   Anthracnose resistance.—Good.
Nut:
   Size.—Large; average length — 1.42″; average diameter in suture plane — 1.22″; average diameter cheek to cheek — 1.42″.
   Uniformity of size.—Not much variation.
   Form.—Somewhat heart-shaped. See FIG. 3.
   Blossom end.—Pointed.
   Basal end.—Flat.
   Thickness of shell.—Thick.
   Ridges.—Rounded.
   Color.—Mottled, 5YR3/2 and 2.5YR3/4 by the Munsell Color Chart for Plant Tissues.
Flowering habit:
   Age at which trees start producing catkins.—Early, about 3-4 years.
   Number of catkins produced.—Abundant.
   Age at which trees start producing pistillate flowers.—Early, about 4-5 years but the flower number varies with the age of the tree.
   Number of pistillate flowers produced by young trees.—Average.
   Lateral shoots producing pistillate flowers.—None.
   Number of pistillate flowers per inflorescence.—2 to 4.
Flower season: Flowers typically in May in Indiana. There are probably 1-million pollen per catkin. Female flowers are about 1/16″ long and grow to two “pollen pick up points” which subsequently break apart. Pollen exists as “dust” which is not feasible to quantitate.
Nut crop:
   Bearing.—Biennial.
   Ripening period.—Average — early October.
   Evenness of maturity (period between first and last nuts are ready for harvest).—Even.
   Quality.—Good.
   Distribution of nuts on tree.—Throughout.
Genetic method of identification DNA “fingerprint” for identification of Beineke 13:

DNA was isolated from the leaves of Beineke 13. For purposes of DNA fingerprinting, eleven highly polymorphic loci from a suite of microsatellites developed by Woeste et al. (2002) were chosen. Microsatellites sizes were checked against previously published standards and verified by a second independent analysis. The “fingerprint” is the collection of microsatellite allele sizes at each locus for “Beineke 13”.

DNA was isolated from the leaves of 4 black walnut trees obtained from Walter Beineke using CTAB extraction buffer (50 mM TRIS-HCL, pH 8.0, 20 mM EDTA, pH 8.0, 0.7 M NaCl, 0.4 M LiCl, 2% SDS, 2% CTAB, nd 1% PVP). After isolation the DNA from each tree was quantified and diluted with nanopure distilled water to a final concentration of 5 ng/microliter. The samples were stored in 96-well plates at −20 degrees C.

For purposes of DNA fingerprinting, eleven highly polymorphic loci from a suite of microsatellites developed by Woeste et al. (2002) were chosen. Amplification of each locus was performed with an MJ Research Tetrad Thermocycler (Waltham, Mass.) using 10 microliter reactions in 96-well plates. The PCR reaction mix contained 2 microliter of the aforementioned black walnut DNA, 5 microliter Sigma Taq ReadyMix (Sigma Aldrich, St. Louis, Mo.), 0.4 microliter of a 20 pmol mixture of forward and reverse fluorescence labeled primer, and 3 microliter PCR grade water supplied with the Sigma ReadyMix. PCR amplification was for 30 cycles of 94 degrees C. for 20 sec, 55 degrees C. for 30 sec, and 72 degrees C. for 1 min. All primers were annealed at 55 degrees C. The products were then held at 4 degrees C. until aliquots could be loaded into 6% Long Ranger (polyacrylamide) denaturing gels (BMA, Rockland, Me.). For each individual 0.5 microliter PCR product was added to 0.75 microliter blue dextran and 0.25 microliter of CXR 350 bp Ladder Standard (Applied Biosystems, Inc., Foster City, Calif.) in a new 96-well plate. The samples were denatured for 2 min at 95 degrees C. and loaded onto a CAL96 96-well laminated membrane comb (The Gel Company, San Francisco, Calif.). Electrophoresis was at 3,000 V, 60 mA, 200 Watts, 50 degrees C. for 2 hours using an ABI 377 (Perkin Elmer) with 36 cm plates and 0.2 mm spacers. The resulting data was analyzed using ABI's GeneScan 3.1.2 and Genotyper 2.5 (Perkin Elmer). Microsatellite sizes were checked against previously published standards and verified by a second independent analysis. The “fingerprint” is the collection of microsatellite allele sizes at each locus for each tree.

| | | |
|---|---|---|
| Locus | Forward | Reverse |

| | | |
|---|---|---|
| WGA6 | CCATGAAACTTCATGCGTTG | CATCCCAAGCGAAGGTTG |
| WGA24 | TCCCCCTGAAATCTTCTCCT | TTCTCGTGGTGCTTGTTGAG |
| WGA27 | AACCCTACAACGCCTTGATG | TGCTCAGGCTCCACTTCC |
| WGA32 | CTCGGTAAGCCACACCAATT | ACGGGCAGTGTATGCATGTA |
| WGA72 | AAACCACCTAAAACCCTGCA | ACCCATCCATGATCTTCCAA |
| WGA89 | ACCCATCTTTCACGTGTGTG | TGCCTAATTAGCAATTTCCA |
| WGA90 | CTTGTAATCGCCCTCTGCTC | TACCTGCAACCCGTTACACA |
| WGA97 | GGAGAGGAAAGGAATCCAAA | TTGAACAAAAGGCCGTTTTC |
| WGA69 | TTAGTTAGCAAACCCACCCG | AGATGCACAGACCAACCCTC |
| WGA76 | AGGGCACTCCCTTATGAGGT | CAGTCTCATTCCCTTTTTCC |
| WGA82 | TGCCGACACTCCTCACTTC | CGTGATGTACGACGGCTG |

The best interpretation of the current data indicates that the probability that any other black walnut tree would have the collection of microsatellite allele sizes listed is estimated to be less than 3×10⁻¹⁴.

Sizes (bp) of microsatellites at 11 loci used to fingerprint “Beineke 13” (2 alleles at each locus).

Microsatellites used to fingerprint Beineke 13:

| WGA6 | | WGA24 | | WGA27 | | WGA32 | |
|---|---|---|---|---|---|---|---|
| 142 | 142 | 236 | 238 | 219 | 223 | 189 | 189 |
| WGA72 | | WGA89 | | WGA90 | | WGA97 | |
| 147 | 149 | 199 | 199 | 162 | 162 | 161 | 171 |

| WGA69 | | WGA76 | | WGA82 | |
|---|---|---|---|---|---|
| 172 | 176 | 230 | 234 | 166 | 180 |

### DOCUMENTS CITED

Beineke, Walter F. (1989) Twenty years of black walnut genetic improvement at Purdue University North. J. Appl. For. 6:68-71.

Woeste, K., Burns, R., Rhodes, O., and Michler, C. (2002) Thirty polymorphic nuclear microsatellite loci from black walnut. Journal of Heredity. 93:58-60.

I claim:

1. new and distinct variety of black walnut tree named “Beineke 13” substantially as illustrated and described, which has excellent timber quality, extremely rapid growth rate, and excellent central stem tendency.
